(19) United States
(12) Patent Application Publication
(10) Pub. No.: US 2019/0223416 A1
(43) Pub. Date: Jul. 25, 2019

(54) CANINE GENOME EDITING

(71) Applicant: Cotyledon, LLC, Darien, CT (US)

(72) Inventor: Matthew Lesko, Darien, CT (US)

(21) Appl. No.: 16/254,260

(22) Filed: Jan. 22, 2019

Related U.S. Application Data
(60) Provisional application No. 62/620,558, filed on Jan. 23, 2018.

(51) Int. Cl. A01K 67/027

(52) U.S. Cl. CPC A01K 67/0275; A01K 2217/056; A01K 2227/10

(57) ABSTRACT

A genetically modified canine has at least one edited chromosomal sequence. The edited chromosomal sequence is insulin-like growth factor 1 gene (“IGF-1”). The IGF-1 gene contains intronic splicing efficiency regions. The individual intronic splicing efficiency regions are altered individually or as a set order to change the IGF-1 gene.

### CROSS-REFERENCE TO RELATED APPLICATION

This application claims priority to and the benefit of U.S. Provisional Application No. 62/620,558, entitled CANINE GENOME EDITING, filed on Jan. 23, 2018, the entire contents of which is incorporated herein by reference.

### BACKGROUND

Canis lupus familiaris, better known as the domestic dog, come in a variety of different shapes and sizes. For example, some dogs, such as the Great Dane, can stand around 28-30 inches tall. In some extreme cases, Great Danes have exceeded 40 inches in height. As to weight, the English Mastiff can weigh as much as 250 pounds, with extreme cases passing 300 pounds. At the other extreme is the Chihuahua, which can weigh between 4-6 pounds and only stand 6-10 inches tall.

As such, different breeds of domestic dog can vary significantly in height and weight. Breeders of domestic dogs have attempted to selectively crossbreed certain dogs so as it to obtain a desired outcome. For example, one of the most common and popular domestic dogs in the United States is the Golden Retriever, which is well-known for its favorable disposition, high trainability, and excellent behavior. However, the Golden Retriever is generally considered a larger dog, with a weight that can exceed 50 or more pounds. As such, breeders have attempted to crossbreed larger and popular breeds, such as the Golden Retriever with smaller dogs, to obtain a dog that has all the benefits of the Golden Retriever, but in a smaller package.

Nevertheless, the result of this crossbreeding is generally mixed. Breeders do not have absolute control over what the outcome of this crossbreeding process will produce. Additionally, crossbreeding creates a certain variance in outcome, wherein one dog produced from the crossbreeding has all the qualities the breeder is looking for, while the other dog may not have all of the same qualities.

One common gene shared by all domestic dogs includes IGF-1. It has generally been observed that smaller breeds of dogs have a variation in the genetic structure of the IGF-1 gene that reduces overall size, and can lead to overall lower serum levels of the IGF-1 protein product. Conversely, larger breeds of dogs have a genetic structure of the IGF-1 gene that results in a larger dog.

### SUMMARY

The present invention generally relates to genetically modified canines, canine cells or canine embryos having at least one edited chromosomal sequence. In particular, the invention relates to editing a chromosomal sequence of insulin-like growth factor 1 (“IGF-1”) in the canine, canine cell or canine embryo.

A genetically modified canine has at least one edited chromosomal sequence. The edited chromosomal sequence may be an IGF-1 gene (IGF1). The IGF-1 gene has a plurality of individual single nucleotide polymorphisms (“SNPs”). Individual SNPs can change the efficiency of gene transcription, leading to a change in transcribed IGF-1 protein levels in the animal.

In one embodiment, the present disclosure describes a nucleic acid for modifying the genomic sequence of a canine, the nucleic acid being selected from the group consisting of SEQ ID No. 1-SEQ ID NO. 17.

In another embodiment, the present disclosure describes a method of modifying the genomic sequence of a canine, the method including a step of transfecting a cell of Canis familiaris with a gene editing device, the gene editing device comprising a sequence for modifying a sequence of an IGF-1 gene.

In another embodiment, the present disclosure is directed to a genetically modified canine comprising at least one edited chromosomal sequence, wherein the edited chromosomal sequence is an IGF-1 gene.

Further objects, features and advantages of this invention will become readily apparent to persons skilled in the art after a review of the following description, with reference to the drawings and claims that are appended to and form a part of this specification.

### BRIEF DESCRIPTION OF THE DRAWINGS

FIG. 1 is a schematic view of the canine IGF-1 gene;

FIG. 2 is a schematic view of a plasmid for use in a gene editing method in accordance with one embodiment of the present disclosure; and

FIG. 3 is a schematic view of another plasmid for use in a gene editing method in accordance with the principles of the present disclosure.

### DETAILED DESCRIPTION

A genetically modified canine has at least one edited chromosomal sequence. The edited chromosomal sequence is insulin-like growth factor 1 (“IGF-1”) gene (IGF1). It should be understood that in addition to being a canine that has at least one edited chromosomal sequence, instead of a canine, the edited chromosomal sequence may be present within a canine embryo or a canine cell.

The gene IGF-1 is conserved from invertebrates through humans. The gene product is a small signaling protein that is transported by the circulatory system. Its primary role is to bind and activate the IGF-1 receptor, a transmembrane protein expressed on the surface of cells, which primarily plays a role in growth of the organism, but also has anabolic functions, including maintenance and healing of cells, tissues, and organs. Lower levels of circulating IGF-1 protein have been correlated with longer animal life, and these functions may play a role in this outcome.

In canines, IGF-1 has been identified as a primary determinant of adult animal size. Large breed dogs tend to have a greater amount of IGF-1 protein in their blood serum than do smaller breed dogs. In many cases, this disparity in serum IGF-1 levels has a genetic cause, with single nucleotide polymorphisms (SNPs) in the IGF-1 gene playing a role.

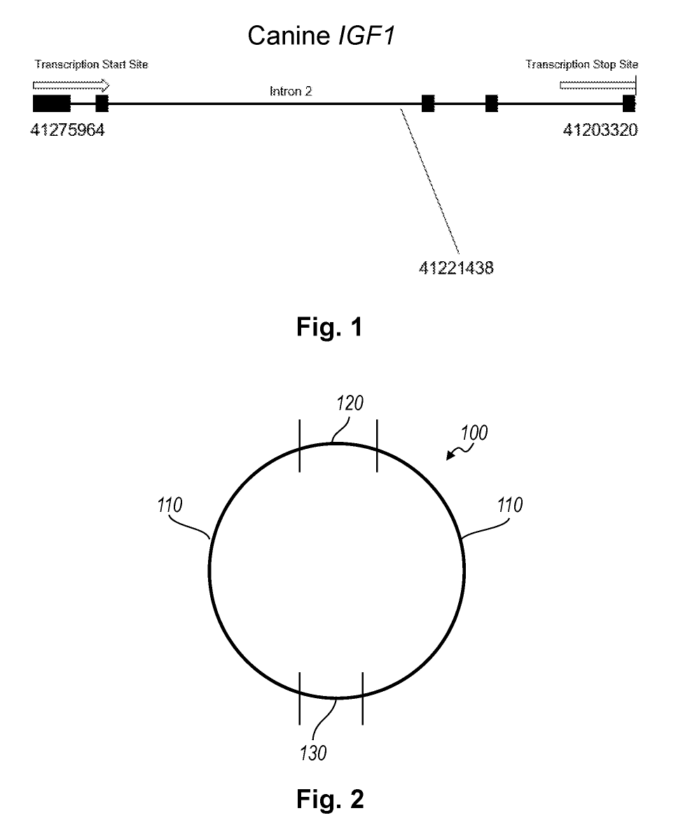

The canine IGF-1 gene (FIG. 1) includes five exons and four introns, and encodes two isoforms of the IGF-1 protein. The IGF-1 gene extends from base 41203320 to base 41275964 on the complement strand of canine chromosome 15, according to the reference genome CanFam3.1. It is noted that all chromosomal locations mentioned herein are done with reference to CanFam3.1.

In silico data demonstrates that certain regions of the gene are statistically correlated to small or large dog size. The context for base pair 41221438 on the (+) strand of chromosome 15 is in a stretch having the sequence GCCAGGCCC, wherein base pair 41221438 is the A in smaller animals. For larger animals, this sequence is instead GCCGGGCCC. Base pair 41221438 corresponds to the second intron in the IGF-1 gene. Without wishing to be bound by any theory, the sequence GGGCCC is associated with DNA bending and flexibility, as well as RNA bending and flexibility, which can increase the efficiency of gene transcription and as a result increase the amount of gene product (that is, the IGF-1 protein). It is associated with RNA splicing efficiency enhancers, and may itself be considered a splicing enhancer sequence. The GGGCCC sequence is also associated with the binding of AP-2 family transcription factors, which contain a transactivation domain, which increases gene expression and is associated with cell proliferation and growth.

As used herein, the term “splicing enhancer” or “splicing enhancer sequence” may refer to a nucleic acid sequence that, directly or indirectly, increases the amount of transcript from a gene in a cell. Intronic splicing enhancers and exonic splicing enhancers are known in the art. Intronic splicing enhancers have been investigated by Wang et al. in Nat. Struc. & Molec. Biol. 19, 1044-1052, for example, the contents of which are incorporated herein by reference.

Likewise, the term “splicing repressor” or “splicing suppressor” refers to an element that has the opposite effect; that is, its presence in a sequence results in a lower level of transcript. Insertions, deletions, and different bases may all act as splicing repressors or suppressors.

In one aspect, a gene editing device may be used to introduce a splicing enhancer sequence into the genome of the canine. In another aspect, a gene editing device may be used to introduce a splicing repressor or suppressor sequence. In some aspects, the gene editing device may alter a genomic sequence to contain a known SNP which has been associated with a desired phenotype.

The nine-base sequences GCCAGGCCC and GCCGGGCCC are core sequences for editing of the canine IGF-1 gene. A core sequence may be used on its own (that is, the 5′ end of the core sequence may be the 5′ end of the nucleic acid, and the 3′ end of the core sequence may be the 3′ end of the nucleic acid), or it may be extended in either the 5′ direction, or in the 3′ direction, or in both directions, so long as the core sequence itself is both present and intact. For example, GCCAGGCCC is a core sequence for the 15mer CCAGCCAGGCCCTGG (SEQ ID NO. 1), which extends SEQ ID NO. 1 three bases in both the 5′ direction and the 3′ direction. Likewise, the 15mer CCAGCCGGGCCCTGG (SEQ ID NO. 8) has GCCGGGCCC as a core sequence.

The nucleic acids of SEQ ID NO. 1 and SEQ ID NO. 8 as disclosed herein, may be effective to modulate the level of IGF-1 protein expressed by a target animal as they alter the genome from a known high IGF-1 expression genotype to low, or vice versa. However, this region of the genome may be edited with other features that will result in an increase or in a decrease of gene expression. For example, SEQ ID NO. 2 is a core sequence 15mer CCAAAAAAAAAA which may be used to introduce a null sequence at a location of a user's choosing. This null sequence may tend to interfere with transcription, such as by decreasing transcriptional efficiency, and thus may be substantially as effective as a transcriptional suppressor as editing to the SNP including GCCAGGCCC. Likewise, CCGTAAAAAAAATGG (SEQ ID NO. 3) and CCAGAAAAACCCTGG (SEQ ID NO. 4) may be used as transcriptional suppressor elements.

In a similar way, splicing enhancer elements may be used in order to yield an increase in IGF-1 expression. Some G/C-rich sequences, including GGGCCC, have been associated with an increase in transcription levels and/or efficiency. In place of a nucleic acid having a core sequence corresponding to GCCGGGCCC, sequences such as CCAGGGGGGCCCTGG (SEQ ID NO. 5), CCAGCCGGCCGGTGG (SEQ ID NO. 6), and CCAGCGCGGCGGTGG (SEQ ID NO. 7) may be used to alter the target genomic region to increase IGF-1 serum levels.

In one embodiment for editing the genome of a canine cell to decrease the size of the resulting adult animal, a nucleic acid to be incorporated into the chromosomal DNA may include a core sequence defined by all or any of AAGACTCTCGTTCTGTTCGCCAGCCAGGCCCTGGCAAGCTGAGACTTGGCC (SEQ ID NO. 9), as long as the core sequence includes the A found at position 26 of the nucleic acid. For example, the core sequence used could be TTCGCCAGCCAGGCCCTGGCA (SEQ ID NO. 10) or AGCCAGGCCCTGGCAAGCTGAGACT (SEQ ID NO. 11). In another aspect, the A at position 26 may instead be C, or may be T.

In one embodiment for editing the genome of a canine cell to increase the size of the resulting adult animal, a nucleic acid to be incorporated into the chromosomal DNA may include a core sequence defined by all or any of AAGACTCTCGTTCTGTTCGCCAGCCGGGCCCTGGCAAGCTGAGACTTGGCC (SEQ ID NO. 12), as long as the core sequence includes the G found at position 26 of the nucleic acid. For example, the core sequence used could be TTCGCCAGCCGGGCCCTGGCA (SEQ ID NO. 13) or AGCCGGGCCCTGGCAAGCTGAGACT (SEQ ID NO. 14).

A person of ordinary skill in the art will appreciate that when a sequence is specified and directed to the (+) strand of the chromosomal DNA, as the DNAs of SEQ ID NOs. 1-16 are, that a DNA complementary to said sequence may also be employed in order to modify the (−) strand. A person of ordinary skill in the art will likewise appreciate that, should a nucleic acid longer than a 15mer be desired, that the nucleic acids of any of SEQ ID NOs. 2-7 can be extended in a manner similar to that which gives rise to SEQ ID NO. 9 and SEQ ID NO. 12.

In order to increase the expected adult size of a canine, or to decrease the expected adult size of a canine, a nucleic acid construct bearing a region for modification of at least one strand of chromosome 15 in the intronic splicing efficiency region around base pair 41221438 of the canine genome may be introduced to a canine cell. In another aspect the modification may be to a different portion of the second intron of the canine IGF-1 gene IGF1. In another aspect, the modification may be to another intron of canine IGF1. Particularly, the cell may be a cell of a canine embryo, which may then be implanted in the uterus of a surrogate mother and allowed to gestate.

Because of the affected region as described above, it is within the scope and spirit of this disclosure to make any other modification by a gene editing device or method that increases or decreases the expression level of IGF-1 protein, or the quantity of mRNA transcript corresponding to the IGF-1 gene in order to influence the size of an adult animal arising from such a change. This is inclusive of making changes to the gene sequence in order to increase or decrease translation efficiency, or to influence the epigenetic characteristics of the IGF-1 gene, either by sequestration or presentation of the chromosomal DNA for transcription, or by covalent modification of the chromosomal DNA, is in the spirit of this disclosure. In some embodiments, the gene editing method may introduce a change such that the serum level of IGF-1 protein in the adult animal is increased or decreased up to about 60% of wild type, and up to about 50% of wild type, or about 70%, or about 90%, or about 100%, or about 50% to about 100% inclusive, or more than about 100%, of wild type.

A genetic splicing device, or gene editing device, may be utilized so as to edit the IGF-1 gene. The genetic splicing device may be any suitable genetic splicing device or methodology. For example, the genetic splicing device or methodology could be CRISPR, TALENs, or zinc finger nucleases. CRISPR is an abbreviation of Clustered Regularly Interspaced Short Palindromic Repeats. CRISPR is a family of DNA sequences in bacteria. Crispr-Cas, including Cas9, is a complex set of enzymes and RNA-based genetic guides that together finds and edits DNA. For background as to how CRISPR works, viruses work by taking over a cell and using the cells biological machinery to replicate until the cell is destroyed. Bacteria have evolved in a way so as to be able to fight viruses. If a bacteria survives a viral attack, the bacteria incorporates portions of the viral genomic sequence into its own genomes, which allows the bacteria to better defend itself from a viral attack from a similar virus by using the viral genomic sequence, in the form of RNA, as a complementary guide for the Cas effector nuclease, which in some cases may be Cas9. CRISPR essentially utilizes the same ability to modify the bacteria of a cell to modify the genetics of a cell. By taking advantage of endogenous DNA repair machinery, these reagents can be used to precisely alter the genomes of higher organisms. CRISPR is described in U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641, all of which are hereby incorporated by reference in their entirety. In another embodiment, a side directed mutagenesis (SDM) method may be employed in order to introduce the modified sequence to the genomic DNA. Examples of SDM methodologies that may be utilized to make such alterations include, for example, the diletto perfetto methodology.

In a CRISPR system, in order to allow the effector nuclease (or Cas) to identify and cut a DNA sequence which can be exploited for integration into the genome, a 2-6 base stretch of DNA known as a protospacer adjacent motif (PAM) may be employed. Use of a PAM improves or is necessary for accurate incorporation. For Cas9, the PAM is represented by NGG, where N can be any of the four main nucleobases. The PAM is appended to the 3′ end of a core sequence, or of another sequence for insertion. For effector nucleases other than Cas9, this sequence may not be NGG. A core sequence for modifying canine IGF-1 as disclosed herein may be appended or synthesized with any PAM at its 3′ end as is known in the art.

It will be appreciated that if a CRISPR system is employed, the nucleic acid that delivers the modified sequence may be delivered to the cell on the same molecule that encodes the CRISPR system, or multiple nucleic acids may instead be employed.

Gene editing by CRISPR may proceed by homology-directed repair (HDR), or non-homologous end joining (NHEJ), or both. In some embodiments, the core sequence may be provided on a nucleic acid designed to primarily proceed by HDR, and in other embodiments, incorporation into the chromosomal DNA may instead primarily proceed by NHEJ. In NHEJ, protein factors re-ligate broken DNAs strand either directly or by including nucleotide insertions or deletions: in the case of the present application, including the altered core sequence. In contrast, HDR uses a homologous repair template to precisely repair the double stranded break in the chromosomal DNA.

In an embodiment where IGF-1 expression levels are to be decreased, such as to generate a relatively smaller animal, and an intron is the region targeted for modification, a gene editing method that functions either by HDR, or by NHEJ, or both, will be effective to result in the desired outcome. Relative to HDR, NHEJ is error prone, but these errors may be acceptable if they have an end result of decreasing expression level of the protein through relatively stochastic sequence modifications in the intron.

In one embodiment, the core sequence can be provided on a small circular plasmid which, in its entirety, or nearly in its entirety, corresponds to canine genomic DNA sequence. One example of such a plasmid is SEQ ID NO. 15, which is 1824 bases, and which can be linearized by Cut1 guide RNA having sequence GTGGGTGCCTCATAGTTGAGNGG (SEQ ID NO. 16) and Cut2 guide RNA having sequence GGGACTATAAATTAGAGGAANGG (SEQ ID NO. 17.)

The plasmid and the two guide-RNAs are delivered with a CRISPR/Cas9 system. The guide-RNA/Cas9 complex is recruited to the specific complementary sequence site by complementary base pairing and the Cas9 protein makes a double stranded break in the plasmid to linearize it. Likewise, the guide-RNA/Cas9 complexes invade the chromosomal DNA near the site of the base to be altered (in this embodiment, about 100 bases in either direction), creating a double strand break that can be repaired, using the linearized plasmid as a template.

FIG. 2 schematically illustrates a nucleic acid construct 100 for use in an HDR-based gene editing workflow. The construct 100 may be a plasmid that includes a first editing sequence 120 and a second editing sequence 130, flanked by regions of homology 110. The regions of homology may have perfect or near-perfect sequence complementarity or homology to regions of the canine genome, including that which has been published as CanFam3.1, surrounding the base or bases to be changed. In one aspect, the first editing sequence 120 may include sequences such as SEQ ID NO. 8 or SEQ ID NO. 9. In one aspect, the second editing sequence may include sequences such as SEQ ID NO. 16 and SEQ ID NO. 17.

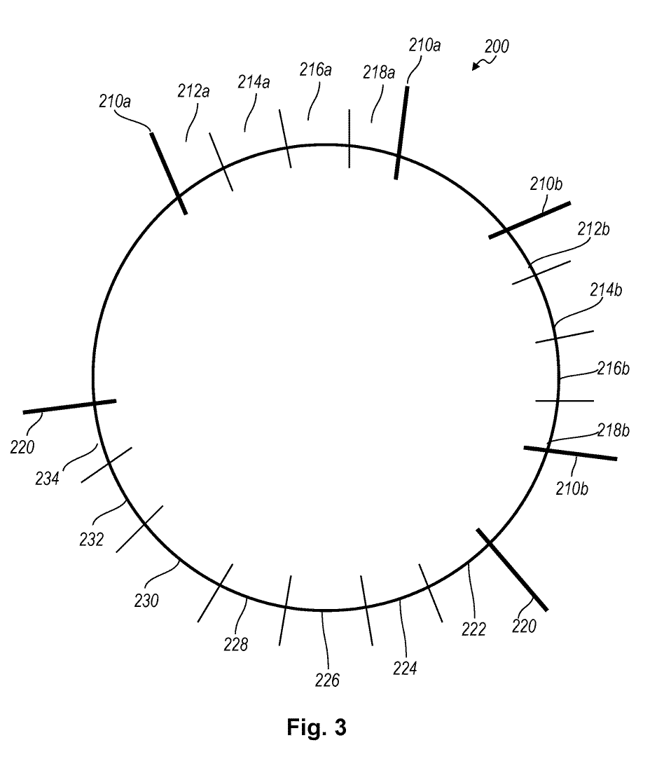

FIG. 3 schematically illustrates another nucleic acid construct 200 for use in a CRISPR gene editing workflow. A person of ordinary skill will appreciate that some of these elements may be substituted with elements of similar function, or may be eliminated, and other elements not listed here may be present in the plasmid. The plasmid 200 as illustrated includes two guide RNA regions 210a and 210b, although a plasmid with more than two such regions may be employed. The guide RNA regions 210a and 210b may include, in a 5′ to a 3′ direction, a promoter 212a/212b, which may be a U6 promoter; a guide RNA sequence 214a/214b, which may be about or exactly 20 nucleotides in length; a guide RNA scaffold 216a/216b; and a termination sequence 218a/218b. The plasmid 200 also includes a CRISPR machinery region 220, which may include elements such as a promoter 222, which may be a constitutive promoter, such as the CAG promoter; a nuclear localization signal 224; a Cas9 variant 226, such as a S. pyogenes Cas9 variant; a 2 A self-cleaving peptide 230, such as a P2A peptide; an expression confirmation region 232, which may be an open reading frame for a reporter protein such as a fluorescent protein, including GFP; and a termination sequence 234.

Gene editing strategies other than CRISPR may be employed for editing of canine IGF-1. Transcription activator-like effector nucleases (“TALENs”) are restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind to practically any desired DNA sequence, so when combined with a nuclease, DNA can be cut at specific locations. The restriction enzymes can be introduced into cells, for use in gene editing or for genome editing in situ, a technique known as genome editing with engineered nucleases. By taking advantage of endogenous DNA repair machinery, these reagents can be used to precisely alter the genomes of higher organisms. TALENs gene editing is described in U.S. Pat. Nos. 9,353,378; 8,440,431; 8,440,432; 8,450,471; 8,586,363; 8,697,853; and 9,758,775, all of which are hereby incorporated by reference in their entirety.

Zinc-finger nucleases are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms. This type of gene editing is described in U.S. Patent Publication No. 2011/0016542A1, which is incorporated by reference in its entirety.

The nucleic acids described, including those bearing core sequences and guide RNAs, may be introduced to the canine cell by any method as is known in the art, such as by transfection. Such transfection may be transient transfection, such as the type achieved by using calcium chloride, a cationic polymer, or a lipid reagent in order to introduce molecules through the membrane of a eukaryotic cell.

### EXAMPLES

Examples of systems, apparatus, and methods according to the disclosed embodiments are described in this section. These examples are being provided solely to add context and aid in the understanding of the disclosed embodiments. It will thus be apparent to one skilled in the art that implementations may be practiced without some or all of these specific details. In other instances, well known process/method steps have not been described in detail in order to avoid unnecessarily obscuring the embodiments. Other applications are possible, such that the following examples should not be taken as definitive or limiting either in scope or setting.

### Example 1

Prophetic increase of a size of a canine of a specific breed. A beagle is generally a smaller breed of canine, with both males and females attaining a healthy adult weight of between 9-11 kg. A larger beagle may be desired. Beagle zygotes can be created in vitro via fertilization or harvested pre-implantation, and be transfected with a first plasmid encoding guide RNAs and a Cas system, such as a Cas9 system, and a second plasmid having a template containing the intronic splicing enhancer sequence GCCGGGCCC at base pairs 41221435-41221443 on the (+) strand of a section of chromosome 15. The modified embryos can be implanted in the uterus of a canine. In this prophetic example, a litter of five puppies may be born, and upon reaching adulthood, attained a healthy weight in a range of between 28-36 kg.

### Example 2

Prophetic decrease of a size of a canine of a specific breed. A mastiff is generally a larger breed of canine, with both males attaining a healthy adult weight of between 73-100 kg, and females attaining a healthy adult weight of between 54-77 kg. A smaller mastiff may be desired. Mastiff embryos can be created in vitro via fertilization or harvested pre-implantation, and be transfected with a first plasmid encoding guide RNAs and a Cas system, such as a Cas9 system, and a second plasmid having a template containing the intronic splicing supressor sequence GCCAGGCCC at base pairs 41221435-41221443 on the (+) strand of a section of chromosome 15. The modified embryos can be implanted in the uterus of a canine. In this prophetic example, a litter of four puppies may be born, and upon reaching adulthood, attained a healthy weight in a range of between 19-30 kg.

As a person skilled in the art will readily appreciate, the above description is meant as an illustration of implementation of the principles of this invention. This description is not intended to limit the scope or application of this invention in that the invention is susceptible to modification, variation and change, without departing from the spirit of this invention, as defined in the following claims.

1. A genetically modified canine comprising at least one edited chromosomal sequence, wherein the edited chromosomal sequence is in an intronic splicing efficiency region, such as an intronic splicing enhancer or suppressor, in an IGF-1 gene.

2. The genetically modified canine of claim 1, wherein the IGF-1 gene is edited using a gene editing device.

3. The genetically modified canine of claim 2, wherein the IGF-1 gene comprises a plurality of individual single nucleotide polymorphisms (“SNPs”), and wherein the gene editing device alters individual SNPs in order to change the IGF-1 gene.

4. The genetically modified canine of claim 3, wherein the heterozygosity of the IGF-1 gene is manipulated.

5. The genetically modified canine of claim 3, wherein the individual SNPs altered are between base pairs 44,212,792 and 44,278,140 on chromosome 15 of the canine.

6. The genetically modified canine of claim 1, wherein the intronic splicing enhancer comprises GGGCCC.

7. A nucleic acid for modifying the genomic sequence of a canine, the nucleic acid comprising a sequence selected from the group consisting of SEQ ID No. 1-SEQ ID No. 17.

8. The nucleic acid of claim 7, wherein the nucleic acid comprises a guide RNA for a genomic editing procedure.

9. The nucleic acid of claim 7, wherein the nucleic acid comprises a plasmid for altering a sequence between base pairs 44,212,792 and 44,278,140 of chromosome 15 of Canis familiaris.

10. A method of modifying the genomic sequence of a canine, the method comprising:
transfecting a cell of Canis familiaris with a gene editing device, the gene editing device comprising a sequence for modifying a sequence of an IGF-1 gene with one of a splicing enhancer or a splicing suppressor.

11. The method of claim 10, wherein the gene editing device comprises a plasmid having sequence homology for a portion of the IGF-1 gene (IGF1), and comprising a sequence for modifying at least one nucleotide of the IGF-1 gene.

12. The method of claim 11, wherein the sequence comprises a sequence for modifying an intron.

13. The method of claim 12, wherein the intron is intron 2 of the IGF-1 gene.

14. The method of claim 12, wherein the gene editing device comprises GGGCCC.

15. The method of claim 10, wherein the gene editing device modifies one nucleotide of the IGF-1 gene.

16. The method of claim 10, wherein the gene editing device creates a deletion in the IGF-1 gene.

17. The method of claim 10, wherein the gene editing device alters the genome in order to increase or decrease a body mass of the resulting organism by at least 20% relative to an average body mass of a breed of said organism.

18. The method of claim 10, wherein the method is effective to modulate the serum level of IGF-1 in the resulting organism.

19. The method of claim 10, wherein the method comprises using one of SEQ ID NO. 1-SEQ ID NO. 17 to modify the genome of the canine.

20. The method of claim 19, wherein the method comprises using one of SEQ ID NO. 1-SEQ ID NO. 8.


SEQUENCE LISTING (17 sequences)

SEQ ID NO: 1 - DNA, 15 bp, Canis lupus
  15mer containing core sequence from Canis lupus
ccagccaggccctgg

SEQ ID NO: 2 - DNA, 15 bp, Artifical sequence
  15mer for null sequence
ccaaaaaaaaaaaaa

SEQ ID NO: 3 - DNA, 15 bp, Artifical sequence
  15mer for use for transcriptional suppressor element
ccgtaaaaaaaatgg

SEQ ID NO: 4 - DNA, 15 bp, Artificial sequence
  15mer for use for transcriptional suppressor element
ccagaaaaaccctgg

SEQ ID NO: 5 - DNA, 15 bp, Artifical sequence
  Element for altering genomic region for IGF-1 serum levels
ccaggggggccctgg

SEQ ID NO: 6 - DNA, 15 bp, Artifical sequence
  Element for altering genomic region for IGF-1 serum levels
ccagccggccggtgg

SEQ ID NO: 7 - DNA, 15 bp, Artifical sequence
  Element for altering genomic region for IGF-1 serum levels
ccagcgcggcggtgg

SEQ ID NO: 8 - DNA, 15 bp, Artifical sequence
  15mer containing core sequence from Canis lupus
ccagccgggccctgg

SEQ ID NO: 9 - DNA, 51 bp, Artifical sequence
  Core sequence for altering animal size
aagactctcgttctgttcgccagccaggccctggcaagctgagacttggcc

SEQ ID NO: 10 - DNA, 21 bp, Artifical sequence
  Core sequence for altering animal size
ttcgccagccaggccctggca

SEQ ID NO: 11 - DNA, 25 bp, Artifical sequence
  Core sequence for altering animal size
agccaggccctggcaagctgagact

SEQ ID NO: 12 - DNA, 51 bp, Artifical sequence
  Core sequence for altering animal size
aagactctcgttctgttcgccagccgggccctggcaagctgagacttggcc

SEQ ID NO: 13 - DNA, 21 bp, Artifical sequence
  Core sequence for altering animal size
ttcgccagccaggccctggca

SEQ ID NO: 14 - DNA, 25 bp, Artifical sequence
  Core sequence for altering animal size
agccaggccctggcaagctgagact

SEQ ID NO: 15 - DNA, 1824 bp, Artifical sequence
  Plasmid for gene editing
tttggtttagttttatttcttattgttttgattcggacacaattttcctggctctagaattaaaaagagagggcaaaacaagcttacgtgaacttgtcataggaacttgaaaagcacctgagttctttgtaaactttttgtcaaagtagaacttctgttaaaccaaactttgcttatacaccaatcgataaagcagacagaagtgaaactgctctggctcaggcgaaggctgggagactggatcttggctttctctcatctctctcatctctcatctcccttgggaggtgcagggcctggtcttctgcactgatattcagccttcatggaggaaaatttgtgaaccactgatccagaagaatccaactaatcaatctggcatatgttcagtctttattcctgacacagaagacaatgtcatattttaaataaaaagtcttacgtttgtataataatgacactattgttgggaatggtgttctgaaagtcaaaatccccatgtatattttgaactccctggttgggaagatcagacccatggaaatggcctgctaccaaactagtttggctgcttcactgcttgaagggccaaccattcatggcatcctctacaaacccaaatgttttcctgatgtttggcacaggagcctggggatccttcctccccataaggcaggtttattgtcattcttagggcagctgggggcagtaattcagtgagggttcatttgtggtctctttgggtggggtctactttctcgcttaggggcaaaccctgtgggtgcctcatagttgagggatttgggagggactaggcagctggccccaattgaagacttagtagtgttttcacttgccattggaggatccacgtgcagaagactctcgttctgttcgccagccaggccctggcaagctgagacttggccagtcccttgggcaatgtaaacaatgtttttttgtttttatagtcttttcctgggactataaattagaggaaagacacaaataggcttacatggttctttgtaatccacaaaggacttctacatactttttgctaagtggttatttcaagaggttgaaggggtcagccagttacgagtagctgatacactttggtttttctcttcgggtggggagaaggtgactcagcctactttgagctctccctgttgtctaccacaggtaatggctgctctctgttgaaactataaacttcttaatgagacataattgggattttttttgttgttgttgtatagtatagattagtagtttgatgttttagaggtggggtagtaaaggtggttccaaaaacatccaaccttggctaactcagaaacaacccatttcaaaattcatgcttatgagttgaagatggagagggataggttttgtaacttcaggacctctttagcatttccatttcatgcatctctactgtaatcttccttctggaggaggacagcaaccttagtcttgggaaggcagaaaacactatgtactgcatattcccttcttgacaactgtcaatagccatgaaactcaccacacatctatttccttttatgaagtagaaagtaatcatcagagcagaaaaaaaaaaaaaaaaagacccacactgggcatctgctttgtcatctttcttagatgagctcagagtttgacatggctcagtctataaaatagctgtgctttttcagtccatagagcaatgaatttatgaacagaaatttaggacttttctgtaatgctccataaagaaagtttaccttatgttcatctattcctttttaatgtgggtgcctcatagttgagggg

SEQ ID NO: 16 - DNA, 23 bp, Artificial sequence
  Synthesized Cut1 guide RNA in which n is a, c, g, or t
gtgggtgcctcatagttgagngg

SEQ ID NO: 17 - DNA, 23 bp, Artificial sequence
  Synthesized Cut2 guide RNA in which n is a, c, g, or t
gggactataaattagaggaangg